(19) United States
(12) Patent Application Publication
(10) Pub. No.: US 2020/0230359 A1
(43) Pub. Date: Jul. 23, 2020

(54) HYPOTUBE CATHETERS

(71) Applicant: Transit Scientific, LLC, Salt Lake City, UT (US)

(72) Inventors: Shawn P. Fojtik, Park City, UT (US); Greg Method, San Francisco, CA (US); Jennifer Arnold, Salt Lake City, UT (US)

(21) Appl. No.: 16/748,541

(22) Filed: Jan. 21, 2020

Related U.S. Application Data
(60) Provisional application No. 62/886,363, filed on Aug. 14, 2019.
(60) Provisional application No. 62/794,976, filed on Jan. 21, 2019.

(51) Int. Cl. A61M 25/01; A61M 25/00

(52) U.S. Cl. CPC A61M 25/0138; A61M 2025/0024; A61M 25/0013; A61M 25/0054

(57) ABSTRACT

A catheter includes a hypotube and a liner. The hypotube may extend substantially along a length of the catheter. The liner may extend along a majority of a length of the hypotube, and may extend along substantially an entirety of the length of the hypotube. The hypotube includes flexibility enhancing features along its length. In addition, the hypotube may include one or more operational features, which may enable various procedures to be performed with the catheter. The liner may be positioned adjacent to an interior surface of a wall of the hypotube, or it may be positioned adjacent to an exterior surface of the wall of the hypotube. Any operational features of the hypotube may be exposed through or beyond a longitudinal extent of the liner or a portion thereof. Methods for manufacturing catheters that include hypotubes and liners are also disclosed.

### CROSS-REFERENCE TO RELATED APPLICATION

Claims for the benefit of the Jan. 21, 2019 filing date of U.S. Provisional Patent Application 62/794,976, titled HYPOTUBE CATHETERS (“the '976 Provisional Application”) and the benefit of the Aug. 14, 2019 filing date of U.S. Provisional Patent Application 62/886,363, titled HYPOTUBE CATHETERS (“the '363 Provisional Application”) are hereby made pursuant to 35 U.S.C. § 119(e). The entire disclosures of the '976 Provisional Application and the '363 Provisional Application are hereby incorporated herein.

### TECHNICAL FIELD

This disclosure relates generally to catheters and, more specifically, to catheters that are defined from hypotubes, or hypodermic tubing. This disclosure also relates to methods for manufacturing catheters and to methods for using catheters.

### RELATED ART

Standard angiography catheters, which are typically manufactured from thermoplastic materials by extrusion processes, have pressure ratings of about 1,050 psi to about 1,200 psi. The pressures that are used in angiography procedures often approach, and sometimes exceed, the pressure ratings of standard angiography catheters. Thus, the use of standard angiography catheters can be risky, as excessive pressures may cause such catheters to fail and, thus, may injury a subject on whom the angiography procedures is being conducted.

Microcatheters typically have thin walls and small diameters to enable them to navigate tiny veins and other vessels within a subject's body. Microcatheters that are used in cardiovascular applications may have outer diameters (ODs) of less than 6 French (F) (2 mm). Neurovascular microcatheters may have outer diameters as small as 2.3 F (0.77 mm). Because of their thin walls and small outer diameters, pushability, trackability, and torqueability requirements have limited the lengths of microcatheters. Typically, the upper limit on the length of a microcatheter is about 150 cm, with some microcatheters—particularly those with smaller outer diameters—being much shorter. In addition, to provide desired levels of pushability, trackability, and torqueability, microcatheters are typically tapered; stated another, way, the wall thickness and/or outer diameter is typically not uniform along the length of the microcatheter.

Hypotubes, or hypodermic tubes, have been used for a variety of purposes, including reinforcing portions of conventional thermoplastic catheters. A hypotube is a thin walled tube formed from a metal or a metal alloy, such as a stainless steel, a nickel titanium alloy (e.g., nitinol, which stands for Nickel Titanium Naval Ordinance Laboratory; etc.), or the like. Hypotubes are useful for a variety of purposes. In the medical device industry, hypotubes have been manufactured in a variety of lengths and with outer diameters ranging from about 0.120 inch (11 gauge) to 0.005 inch (36 gauge). Among other purposes, hypotubes have been used to facilitate the introduction of catheters through a patient's anatomy.

Medical hypotubes are commonly formed from stainless steel (e.g., 304 stainless steel, 316 stainless steel, 316L stainless steel, etc.). While stainless steel hypotubes may enhance the ability of a catheter to glide (e.g., push, track, and torque) through a subject's anatomy, they are known for their tendency to kink, particularly when forced through tortuous paths. Nitinol has also been used to form hypotubes. While nitinol hypotubes are kink-resistant, they are much more expensive to manufacture (particularly in developmental stages and in small quantities) than stainless steel hypotubes, and still lack sufficient flexibility to enable them to advance through many of the bends that are present in a subject's vasculature.

### SUMMARY

A catheter according to this disclosure may include one or more hypotubes and a liner. The catheter may be defined primarily by the one or more hypotubes. The hypotube(s) may include flexibility enhancing features that enable the hypotube to be used to at least partially define the catheter. The liner may be associated with the hypotube(s) in a manner that controls the flow of fluids through the walls of the catheter. The catheter may also include a tip at its distal end, as well as a coupling feature (e.g., a luer lock, etc.) at its proximal end.

In some embodiments, a catheter may consist essentially of a hypotube (e.g., a single hypotube, a series of hypotubes, etc.), a liner, and a coupling feature. In other embodiments, a catheter may consist essentially of a hypotube, a liner, a tip, and a coupling feature; such catheters may include one or more non-essential features. In still other embodiments, a catheter may consist of a hypotube, a liner, and a coupling feature or a hypotube, a liner, a tip, and a coupling feature.

The one or more hypotubes may extend along the entire length of the catheter. Alternatively, one or more hypotubes may extend along substantially the entire length of the catheter. As used in reference to the length of one or more hypotubes relative to the length of a catheter, the term “substantially” indicates that one or more distal features of the catheter may extend slightly beyond a distal end of the hypotube, one or more proximal features may extend slightly beyond a proximal end of the hypotube, and/or small gaps may be present between two or more hypotubes that have been arranged in series along the length of the catheter. Further, the term “substantially,” as used in connection with the length of one or more hypotubes relative to the length of the catheter of which the one or more hypotubes are a part, may be quantified as at least 95% of a length of the catheter, as at least 90% of a length of the catheter, as at least 80% of a length of the catheter, or even as at least 75% of a length of the catheter. In a specific embodiment, the catheter may include a single hypotube.

The flexibility enhancing features of a hypotube may impart the hypotube and a corresponding portion of the catheter with a desired amount of flexibility. The configurations and/or positioning of the flexibility enhancing features may determine the flexibility of the hypotube and the catheter.

The flexibility enhancing features of a hypotube may comprise cuts into or through a wall of the hypotube. In some embodiments, the cuts may extend partially around a circumferential location along the length of the hypotube (e.g., around up to about 95% of the circumference of the hypotube, etc.), normal to a longitudinal axis of the hypotube. These types of cuts are referred to as “circumferential cuts.” U.S. Design patent application No. 29/635,209, filed Jan. 29, 2018 (“the '209 Design application) and U.S. Provisional Patent Application No. 62/623,180, filed on Jan. 29, 2018 (“the '180 Provisional Application), the entire disclosures of which are hereby incorporated herein, disclose embodiments of flexibility enhancing features that comprise circumferential cuts.

Another embodiment of flexibility enhancing feature includes one or more cuts that are oriented helically, or that spiral, around at least a portion of a length of the hypotube (i.e., at a non-perpendicular angle to the longitudinal axis of the hypotube). The length of each cut in a helical series of cuts, as well as the distance between helically adjacent cuts located along a given helical path around the circumference of the hypotube, or the length of each solid region between helically adjacent cuts located along the given helical path, may define the flexibility of a portion of the length of the hypotube. Each helically oriented cut may extend partially around the circumference of the hypotube (i.e., have an arc length of less than 2π, or 360°) (e.g., up 95% of the circumference of the hypotube, or have an arc length of up to about 1.9π, or 342°, etc.). In other embodiments, each helically oriented cut may extend completely around the circumference of the hypotube (i.e., have an arc length of 2π, or 360°, or greater) provided that the helical series of cuts does not facilitate extension of a length of the hypotube upon pulling ends of the hypotube in opposite directions or upon holding one end of the hypotube stationary while pulling on the other end of the hypotube. In some embodiments, a flexibility enhancing feature may be defined by a plurality of helical series of cuts that share the same axis, but are angularly translated around the hypotube.

The flexibility of a portion of the hypotube along which a longitudinal series of flexibility enhancing features is located (i.e., along a length of the hypotube) may be defined, at least in part, on the spacing, or pitch, between longitudinally adjacent flexibility enhancing features (e.g., the longitudinal distance between two longitudinally adjacent circumferential cuts; the distance between locations along an element of the hypotube intersected by longitudinally adjacent portions of one or more cuts oriented along a helical path around the circumference of the hypotube; etc.). A portion of a hypotube that includes shorter spacing between adjacent flexibility enhancing features, or a tighter pitch, may be more flexible than a portion of the hypotube that includes greater spacing between adjacent flexibility enhancing features, or a wider pitch. Conversely a portion of a hypotube that includes greater spacing between adjacent flexibility enhancing features, or a wider pitch, may be stiffer than a portion of the hypotube that includes shorter spacing between adjacent flexibility enhancing features, or a tighter pitch.

Various other factors may also determine the flexibility one or more flexibility enhancing features impart to a portion of a hypotube and, thus, to a corresponding portion of a catheter of which the hypotube is a part. As an example, the flexibility provided by a cut (e.g., a circumferential cut, a helically oriented cut, etc.) or a series of cuts (e.g., a circumferential series of circumferential cuts (i.e., a series of cuts arranged around a circumferential location around the hypotube and along the length of the hypotube, normal to its longitudinal axis); a helical series of helically oriented cuts; etc.) may be a function of the length(s) of the (series of) cut(s) relative to the distance around the circumference, as well as a function of the number of solid sections (e.g., one solid section when a single cut extends around a portion of a circumference of the hypotube; two solid sections between adjacent ends of a series of two cuts extending around different portions of the circumference of the hypotube; etc.) and/or the length of each solid section between circumferentially or helically adjacent cuts. In addition, when helically oriented cuts are used to define flexibility enhancing features of a hypotube, flexibility of a portion of the hypotube may be tailored by positioning cuts of different handedness (i.e., left-handedness, right-handedness), or chirality, adjacent to one another.

In some embodiments, the hypotube and the catheter may have different flexibilities at different locations along their lengths. Different types and/or arrangements of flexibility enhancing features at different locations along the length of the catheter may impart those different locations with different flexibilities. As a nonlimiting example, the flexibility enhancing features of a hypotube located at proximal and intermediate locations along the length of the catheter may impart the proximal and intermediate portions of the catheter with sufficient flexibility to enable these portions to extend along the curves and/or bends of pathways through the body, while imparting the proximal and intermediate portions of the catheter with sufficient stiffness to enable them to transmit a distally oriented pushing force to a distal portion and a distal end of the hypotube and, thus, to a distal portion and a distal end of the catheter. Stated another way, flexibility enhancing features that are to be located along the proximal portion and the intermediate portion of a catheter may impart these portions of the catheter with a desired amount of “pushability.” The flexibility enhancing features of a hypotube located at a distal portion of the catheter may render the distal portion of the catheter even more flexible, imparting the distal end of the catheter with the ability to navigate tortuous vasculature, or “trackability.”

Other types of features may also be defined in a hypotube of a catheter according to this disclosure. As a nonlimiting example, a portion of a length of a hypotube may include features that enable expansion and, optionally, resilient contraction, of a diameter (including, for example, both the inner diameter (ID) and the outer diameter) of the hypotube. Examples of such features are disclosed by U.S. patent application Ser. No. 16/174,205, filed on Oct. 29, 2018 (“the '205 application), U.S. Provisional Patent Application No. 62/735,110, filed on Sep. 22, 2018 (“the '111 Provisional Application), U.S. Design patent application No. 29/625,044, filed on Nov. 6, 2017 (“the '044 Design application), U.S. Design patent application No. 29/670,032, filed on Nov. 13, 2018 (“the '032 Design application), and U.S. Design patent application No. 29/670,041, filed on Nov. 13, 2018 (“the '041 Design application), the entire disclosures of which are hereby incorporated herein. Such expandable features and/or other types of features that are defined in a hypotube may be positioned along at a distal portion of the catheter of which the hypotube is a part (e.g., adjacent to a distal end of the catheter, etc.). Alternatively, or in addition, expandable features or other features defined in a hypotube may be positioned at an intermediate location along the length of the catheter of which the hypotube is a part and/or along a proximal portion of the catheter of which the hypotube is a part.

In a specific embodiment, a catheter according to this disclosure may be most flexible at its distal end, include features that enable radial expansion of the catheter along at least a portion of a distal region, and provide for some flexibility, as well as pushability, along its intermediate and proximal portions.

The liner of a catheter according to this disclosure may control the flow of fluids through the walls of the catheter. In addition, the interior surface of a liner that covers the interior surface of the wall of the hypotube may be smoother, or more lubricious, than the interior surface of the wall of the hypotube. The increased smoothness provided by an interior liner may increase the ease with which a catheter to pass over a guide wire, increase the ease with which other instruments may be introduced into and removed from a subject's body through the catheter, and increase the smoothness with which fluids and other materials flow through the lumen of the catheter. A configuration of the liner (e.g., the material or materials from which it is made, its thickness, its positioning on the hypotube(s), etc.) may dictate how the liner affects the flexibility of the hypotube(s) of the catheter and, thus, the flexibility of the catheter. In some embodiments, the liner may have a configuration that minimizes its impact on the flexibility of the hypotube(s) and, thus, on the flexibility of the catheter, while preventing fluids from flowing through the catheter wall at unintended locations along the length of the catheter. The liner may also have sufficient flexibility, stretchability, and/or resilience to accommodate other features of the hypotube, including, without limitation, features that enable expansion of a portion of the hypotube (e.g., to enable an expandable section to expand, and then facilitate contraction of the expandable portion to its original diameter or substantially to its original diameter (accounting for minor stretching).

The liner may coat an interior surface (i.e., a lumen) of a wall of a hypotube of the catheter, an exterior surface of the wall of the hypotube, or the catheter may include an interior liner on the interior surface of the wall of the hypotube and an exterior liner on the exterior surface of the wall of the hypotube. Each liner may extend along an entire length or substantially an entire length of the catheter (in this context, the term “substantially” has the same meaning as provided previously herein with respect to the portion of the length of a catheter along which a hypotube or a series of hypotubes extends). As another alternative, the liner may include a collection of plugs that fill and seal flexibility enhancing features (e.g., cuts, etc.) along the hypotube (e.g., all of the flexibility enhancing features of the hypotube, selected flexibility enhancing features of the hypotube, etc.) without extending substantially across solid regions of the hypotube (the term “substantially” being used in this context in recognition of the possibility that the material that forms each plug may extend onto surfaces of the solid regions that are located adjacent to a cut within which a plug resides).

A catheter according to this disclosure may have a uniform profile or a substantially uniform profile along its entire length. “Substantially,” as used in reference to the uniform profile of the catheter, may account for acceptable manufacturing tolerances in the outer diameter and outer surface of a hypotube (e.g., in embodiments where the liner of the catheter is located on the interior surface of the wall of the hypotube). In embodiments where the catheter includes an external liner, a “substantially” uniform profile may account for acceptable tolerances in the external liner (e.g., in the wall thickness of the external liner as it is manufactured separately from the hypotube; in variations in the wall thickness of a separately manufactured hypotube as it is introduced over and secured to (e.g., by shrinking, etc.) the hypotube; in variations in the thickness of an external liner that is formed on the outer surface of the wall of the hypotube; etc.). A “substantially” uniform profile may also account for slight variations to the profile that may occur under ordinary conditions (e.g., manufacture, packaging, transportation, storage, use, etc.), during which changes in environment (e.g., temperature, humidity, etc.) may affect the materials from which the catheter, including its hypotube(s) and liner(s), are formed.

In a method for manufacturing a catheter, a hypotube of a desired material and dimensions (e.g., outer diameter, inner diameter, etc.) is selected. Flexibility enhancing features are defined at desired locations along a portion of the hypotube that is to be used to define the catheter. Other features may also be defined at desired locations along the length of the hypotube. Without limitation, flexibility enhancing features and any other features may be defined in the hypotube by laser cutting processes.

Once the flexibility enhancing features and any other features have been defined in the hypotube, a liner may be applied to the hypotube. In some embodiments, the liner may reside on the interior surface of a wall of the hypotube, which defines a lumen of the hypotube. As a nonlimiting example, the liner may comprise a preformed tube with outer cross-sectional dimensions, taken normal to a longitudinal axis of the liner, that enable it to be received by the inner diameter, or lumen, of the hypotube. Such a liner may comprise a material that enables it to expand until an outer surface of the liner abuts and engages the interior surface of the wall of the hypotube (e.g., polytetrafluoroethylene (PTFE), such as the material marketed by The Chemours Company of Wilmington, Del. under the TEFLON® trademark), etc.). Once the liner is positioned within desired locations along the length of the hypotube, the liner may be subjected to processes that will cause it to expand into, or to fit, the interior surface of the wall of the hypotube, thereby securing the liner to the interior surface of the wall of the hypotube.

In other embodiments, the liner may comprise a preformed tube of a contractible, or shrinkable, material (e.g., a heat shrinkable material, such as polyethylene terephthalate (PET); etc.) with a lumen having cross-sectional dimensions, taken normal to a longitudinal axis of the liner, that enable it to receive the outer diameter of the hypotube. The liner may be cut to a desired length, and the hypotube may be introduced into the lumen of the liner. Once the liner is positioned over desired locations along the length of the hypotube, the liner may be subjected to processes that will cause it to contract, or shrink, onto the hypotube, thereby securing the liner to an exterior surface of the wall of the hypotube.

Other techniques for forming internal liners, external liners, and other configurations of liners that reinforce and/or control the passage of fluids through flexibility enhancing features and/or any other features that have been defined in the hypotube may also be used in manufacturing a catheter according to this disclosure.

The hypotube may be cut to a desired length prior to forming flexibility enhancing features and any other features, prior to assembling the liner with the hypotube or forming the liner on the hypotube, or once the liner has been applied to the hypotube. In some embodiments, a taper may be defined at the distal end of the hypotube (e.g., by altering the distal end of the hypotube, etc.) or provided at the distal end of the hypotube (e.g., by coupling a distal tip to the distal end of the hypotube, etc.). Such a taper may, by way of example only, be oriented at an angle of up to about 5° to the longitudinal axis of the hypotube.

A catheter according to this disclosure may also include a tip at its distal end. The tip may be formed or secured to a distal end of a hypotube of the catheter in a manner that mechanically interlocks the tip to the distal end of the hypotube.

Other aspects of the disclosed, subject matter, as well as features and advantages of various aspects of the disclosed subject matter, should become apparent to those of ordinary skill in the art through consideration of the ensuing description, the accompanying drawings, and the appended claims.

### BRIEF DESCRIPTION OF THE DRAWINGS

In the drawings:

FIG. 1 schematically illustrates an example of positioning of a catheter within a body of a subject;

FIG. 2 is a schematic representation of an embodiment of a catheter that includes a hypotube and a liner, with the liner lining substantially all of an interior surface of the wall of the hypotube;

FIGS. 3A, 3B, and 3C respectively show distal, intermediate, and proximal portions of the embodiment of catheter depicted by FIG. 2.

FIGS. 4A, 4B, and 4C are cross-sectional representations of the distal, intermediate, and proximal portions, respectively, of the embodiment catheter depicted by FIG. 2;

FIG. 5 is a schematic representation of another embodiment of a catheter, which includes a hypotube and a liner, with the liner lining portions of an interior surface of the wall of the hypotube and with one or more portions of the interior surface of the wall of the hypotube being exposed beyond a longitudinal extent of the liner or through the liner (e.g., between adjacent sections thereof, in an aperture thereof, etc.);

FIGS. 6A, 6B, and 6C respectively show distal, intermediate, and proximal portions of the embodiment of catheter depicted by FIG. 5;

FIGS. 7A, 7B, and 7C are cross-sectional representations of the distal, intermediate, and proximal portions, respectively, of the embodiment catheter depicted by FIG. 5;

FIG. 8 illustrates an embodiment of a catheter defined from a hypotube that includes an embodiment of flexibility enhancing feature with a plurality of helices;

FIG. 9 is a schematic representation of an embodiment of a catheter that includes a plurality of hypotubes arranged end-to-end, or in a longitudinal series, and a liner that secures the hypotubes in position relative to one another;

FIG. 10 is a schematic representation of another embodiment of a catheter that includes a hypotube and a liner, with the liner lining at least a portion of an exterior surface of the wall of the hypotube;

FIGS. 11A, 11B, and 11C respectively show distal, intermediate, and proximal portions of the embodiment of catheter depicted by FIG. 9;

FIGS. 12A, 12B, and 12C are cross-sectional representations of the distal, intermediate, and proximal portions, respectively, of the embodiment catheter depicted by FIG. 10; and

FIGS. 13 and 14 illustrate embodiments of catheters according to this disclosure that includes tips interlocked with distal ends of hypotubes.

### DETAILED DESCRIPTION

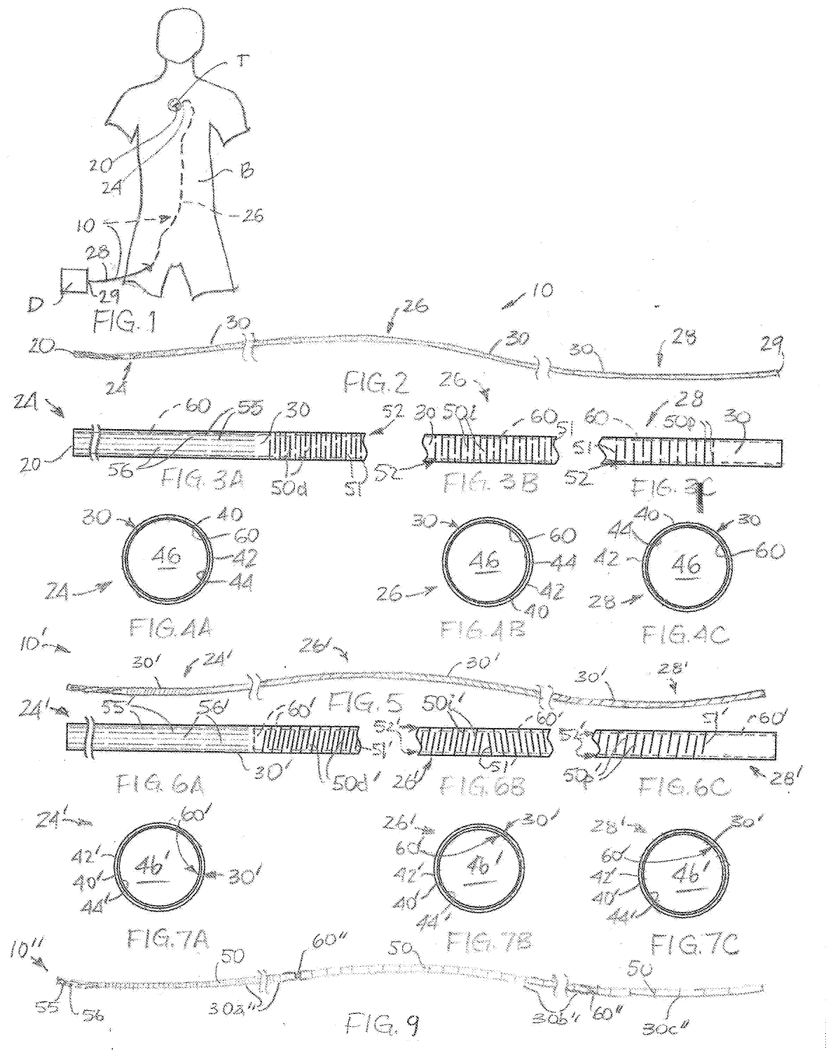

FIGS. 2-4C illustrate an embodiment of a catheter 10 that includes a hypotube 30 and a liner 60. The hypotube 30, which defines an exterior portion of a majority of a length of the catheter 10, extends along substantially an entire length of the catheter 10. The liner 60 defines an interior of a majority of the length of the catheter 10.

Along its length, the catheter 10 includes a distal end 20, a distal portion 24, an intermediate portion 26, a proximal portion 28, and a proximal end 29. As shown in FIG. 1, the distal end 20 of the catheter 10 is capable of being introduced into a body B of a subject and advanced through the body B of the subject (e.g., along a pathway, such as the subject's blood vessels, or vasculature, other vessels, or other tubes or passages) to a target site T, where a procedure is to be performed. Upon advancement of the distal end 20 to the target site T, the procedure that is to be performed may be performed using the distal end 20 or the distal portion 24 of the catheter 10, features on the distal end 20 or the distal portion 24, and/or devices associated with the distal end 20 or the distal portion 24.

The intermediate portion 26 of the catheter 10, which may comprise a majority of the length of the catheter 10, is configured to enable advancement of the distal end 20 and/or the distal portion 24 of the catheter 10 through the subject's body B to the target site T, to reside within the body B of the subject once the distal end 20 and/or distal portion 24 has been advanced to the target site T, to enable further movement of distal end 20 and the distal portion 24 relative to the target site T, and to enable removal of the catheter 10 and anything carried thereby (e.g., tissue samples, debris, devices, etc.) from the body B of the subject. During advancement and removal of the catheter 10, a healthcare professional may hold the intermediate portion 26 at one or more locations to respectively push the catheter 10 into the subject's body and pull the catheter 10 out of the subject's body B.

The proximal portion 28 of the catheter 10 is configured to reside outside of the subject's body B during advancement, use, and removal of the catheter 10. A proximal end 29 of the proximal portion 28 of the catheter 10 may have a configuration that enables it to couple to one or more devices D that are to be used externally by a healthcare professional to perform one or more procedures at a target site T (or a plurality of target sites T) within the subject's body B.

As shown in FIGS. 3A-4C, the hypotube 30 may comprise an elongated tube defined by a wall 40 with an outer surface 42 and an interior surface 44. The interior surface 44 of the wall 40 defines a lumen 46 that extends through a length of the hypotube 30. A hypotube 30 of a catheter 10 (FIG. 2) according to this disclosure may be formed from any of a variety of suitable materials. The material from which the hypotube 30 is formed may be rigid, but have some flexibility when used to form extremely thin structures, such as the wall 40 of the hypotube 30. Examples of such materials include stainless steel (e.g., 304 stainless steel, 316 stainless steel, 316L stainless steel, etc.), nickel-chromium (NiCr) steel, nitinol, cobalt/chromium, other metals and metal alloys, and various plastic materials. The dimensions of the hypotube 30 (e.g., its outer diameter, the thickness of its wall 40, its inner diameter, its length, etc.) may be suitable for one or more desired uses of the catheter 10. As examples, the outer diameter of the hypotube 30 may be about 0.005 inch (about 125 μm) to about 0.080 inch (about 2 mm, or 6 F). The inner diameter of the hypotube 30, or the diameter of the lumen 46 of the hypotube 30, may be about 0.002 inch (about 50 μm) to about 0.070 inch (about 1.8 mm). Since a hypotube 30 is used to define the catheter 10, even microcatheter embodiments of the catheter 10 may have outer diameters, wall thicknesses, and inner diameters that are uniform or substantially uniform (i.e., within acceptable tolerances) along their entire lengths. Such catheters 10 may have lengths of 150 cm or more than 150 cm (e.g., 175 cm, 200 cm, etc.) while providing desired or required levels of pushability, trackability, and torqueability.

Flexibility enhancing features 50d, 50i, and 50p of the hypotube 30 are respectively located at the distal portion 44, the intermediate portion 26, and the proximal portion 28 of the embodiment of catheter 10 depicted by FIGS. 2-4C. The flexibility enhancing features 50d, 50i, and 50p may comprise cuts that may extend from the outer surface 42 of the wall 40 of the hypotube 30, partially or completely through a thickness of the wall 40.

The cut that defines a flexibility enhancing feature 50d, 50i, 50p may comprise a circumferential cut or a helically oriented cut. A circumferential cut may be oriented normal to a longitudinal axis of the hypotube 30, and may extend partially around the circumference of the hypotube 30 (e.g., up to about 95% of the circumference of the hypotube, etc.).

Solid regions between ends of adjacent cuts 51 may define a spine 52 of a flexibility enhancing feature 50d, 50i, 50p. The spine 52 may be oriented helically as shown in FIG. 3A, 3B, or 3C or longitudinally. A flexibility enhancing feature 50d, 50i, 50p may include a single spine 52 or a plurality of spines 52 (e.g., two spines 52, three spines 52, four spines 52, etc.), depending upon the arc length each cut 51 that defines the flexibility enhancing feature 50d, 50i, 50p. As each spine 52 is defined at least in part by solid regions of the hypotube 30, each spine 52 may resist bending. The width(s) of the spine(s) 52, the number of spines 52, and the arrangement of spines 52 around the circumference of a flexibility enhancing feature 50d, 50i, 50p may at least partially dictate the column strength of the flexibility enhancing feature 50d, 50i, 50p, or the ability of the flexibility enhancing feature 50d, 50i, 50p to resist compression. The width of each spine 52 and the number of spines 52 around the circumference of the hypotube 30 may contribute to the flexibility/stiffness of the flexibility enhancing feature 50d, 50i, 50p. The orientation of each spine 52 on the flexibility enhancing feature 50d, 50i, 50p may determine the direction(s) in which the flexibility enhancing feature 50d, 50i, 50p may be deflected, or bend. Thus, a flexibility enhancing feature 50d, 50i, 50p may impart the catheter 10 with steerability without adding to the thickness, or outer diameter, of the catheter 10 and without decreasing the size (e.g., inner diameter, etc.) of any lumen extending through the catheter 10.

The orientation of a cut 51 through the through the wall 40 of the hypotube 30 may be perpendicular to a tangent to the outer surface 42 of the wall 40 (i.e., it may extend the shortest possible distance through the wall 40, i.e., straight through the wall). Alternatively, a cut 51 that forms a flexibility enhancing feature 50d, 50i, 50p (each of which may also be referred to as a flexibility enhancing feature 50) may extend through the wall 40 at a non-perpendicular angle to a tangent to the outer surface 42 of the wall 40 (i.e., diagonally).

Each cut 51 may be performed by any of a variety of suitable processes, including, without limitation, by laser cutting techniques. In some embodiments, a laser beam with a nominal width, or kerf, of 0.0012 inch or less may be used. Smaller laser beam widths may be used to cut sharper features. Defocused laser beams with widths of up to about 0.0012 inch may be used to create angled cuts or shaped cuts 51 (e.g., hourglass shaped cuts, etc.), which may define smooth corners at the inner diameter of a catheter 10. The creation of cuts 51 with smooth corners at the inner diameter of a catheter 10 may impart the catheter 10 with internal lubricity, which may enable fluids to flow more freely (e.g., laminar flow, etc.) through a lumen of the catheter 10 and minimize the friction with which other devices, such guide wires, pass through a lumen of the catheter 10.

As FIGS. 3A-3C illustrate, flexibility enhancing features 50d, 50i, 50p may be arranged differently at different locations along the length of the catheter 10 (FIG. 2). As nonlimiting examples, the flexibility enhancing features 50d on a distal portion 24 of the catheter 10 (FIG. 4A) may be positioned closer together (i.e., have a tighter pitch) than the flexibility enhancing features 50i of the intermediate portion 26 of the catheter 10 (FIG. 4B) and the flexibility enhancing features 50p of the proximal portion 28 of the catheter 10 (FIG. 4C). Such an arrangement may render the part of the distal portion 24 where the flexibility enhancing features 50d are located to be more flexible and, thus, to have greater trackability than the parts of the intermediate portion 26 and the proximal portion 28 where flexibility enhancing features 50i and 50p are respectively located. The flexibility enhancing features 50i and 50p of the intermediate portion 26 and the proximal portion 28 may render these portions of the catheter 10 more rigid than the distal portion 24 and, thus, impart these portions of the catheter with more pushability than the distal portion 24. Additionally, the flexibility enhancing features 50p of the proximal portion 28 may be spaced further apart from one another than the flexibility enhancing features 50i of the intermediate portion 26, making the proximal portion 28 more rigid and, therefore, imparting the proximal portion 28 with greater pushability than the intermediate portion 26.

In addition to the spacing, or pitch, between adjacent flexibility enhancing features 50d, 50i, 50p, other factors regarding the lengths, orientations, and positioning (e.g., spacing between a series of circumferentially oriented or helically oriented flexibility enhancing features, etc.) may at least partially contribute to the flexibility and/or rigidity of a part of the hypotube 30.

Without limitation, the flexibility enhancing features 50d, 50i, and 50p may be arranged in a manner similar to that disclosed by the '209 Design application and the '180 Provisional Application. While FIGS. 3A-3C depict flexibility enhancing features 50d, 50i, 50p that comprise circumferential cuts, the hypotube 30 may include other embodiments of flexibility enhancing features 50 (e.g., helically or spirally oriented cuts, etc.—see, e.g., FIGS. 6A-6C and 8) in place of or in addition to the circumferential cuts shown in FIGS. 3A-3C.

In addition to including flexibility enhancing features 50, a hypotube 30 of a catheter 10 (FIG. 2) according to this disclosure may include operational features 55, 56, as depicted by FIG. 4A, which may enable the hypotube 30 to be used to perform a particular procedure at a target site T (FIG. 1) within the body of a subject. Without limitation, the operational features 55, 56 may respectively comprise cuts that are generally longitudinally oriented and generally longitudinally oriented rotatable struts defined between the cuts, which together may define an expandable section along the length of the hypotube 30 (e.g., along a portion of the hypotube 30 comprising at least part of a distal portion 24 of the catheter 10, etc.), as further disclosed by the '205 application, the '044 Design application, the '032 Design application, and the '041 Design application.

With returned reference to FIGS. 4A-4C, the hypotube 30 may have any suitable dimensions. Without limitation, the wall 40 of the hypotube may have a thickness (i.e., a distance along a radius of the hypotube 30) of about 0.0015 inch (about 38 μm) to about 0.006 inch (about 150 μm); the inner diameter of the hypotube 30, or the diameter of the lumen 46 of the hypotube 30, may be about 0.002 inch (about 50 μm) to about 0.070 inch (about 1.8 mm); and the outer diameter of the hypotube 30 may be about 0.005 inch (about 125 μm) to about 0.080 inch (about 2 mm).

As FIGS. 4A-4C depict, and as is apparent from FIGS. 2 and 3A-3C, the hypotube 30 defines an exterior of the catheter 10 (FIG. 2), while the liner 60 defines an interior of the catheter 10. In such an embodiment, the liner 60 may comprise a polymer that lines the interior surface 44 of the wall 40 of the hypotube 30. In a specific embodiment, the liner 60 may comprise polytetrafluoroethylene (PTFE). More specifically, the liner 60 may comprise an expandable PTFE (ePTFE) tube (e.g., the AEOS® expandable PTFE (ePTFE) tubing available from Zeus Industrial Products, Inc., of Orangeburg, S.C., etc.) that has been expanded to abut the interior surface 44 of the wall 40 of the hypotube 30 and to fit the inner diameter of the lumen 46 defined by the interior surface 44 of the wall 40 of the hypotube 30. Such a liner 60 may have a thickness of about 0.00025 inch (about 6.35 μm) to about 0.0005 inch (about 12.7 μm). An interior surface 64 of the liner 60 defines the inner diameter of the catheter 10.

In the embodiment illustrated by FIGS. 2-4C, the liner 60 extends along substantially an entire length of the catheter 10. Such a liner 60 may line an entire interior surface 44 of the hypotube 30.

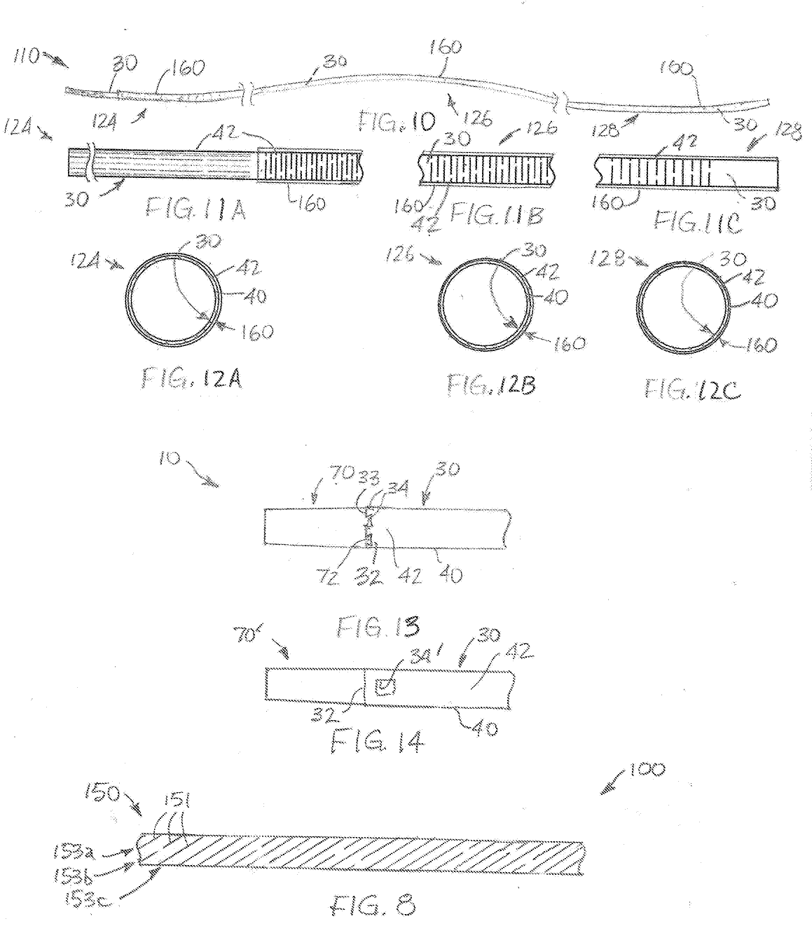

FIGS. 5-7C depict an embodiment of catheter 10′ with a liner 60′ on only a portion of the interior surface 44′ of the wall 40′ of a hypotube 30′. As illustrated by FIGS. 7A, 7B, and 7C, the liner 60′ resides on the interior surface 44′ of the wall 40′ of the hypotube 30′ along a proximal portion 28′ of the catheter 10′ (FIG. 7C) and along its intermediate portion 26′ (FIG. 7B), but the liner 60′ does not reside on the interior surface 44′ of the wall 40′ of the hypotube 30′ along at least part of the distal portion 24′ of the catheter 10′ (FIG. 7A). More specifically, the interior surface 44′ of the wall 40′ of the hypotube 30′ may be exposed at a location that includes operational features 55′, 56′ (FIG. 6A) (e.g., the cuts and rotating struts, respectively, of an expandable section of the hypotube 30′, as disclosed by the '205 application, the '032 Design application, and the '041 Design application; etc.). A liner 60′ may, however, cover other areas of the interior surface 44′ of the wall 40′ of the hypotube 30′.

The flexibility enhancing features 50d′, 50i′, and 50p′ may be arranged along one or more helical, or spiral, paths around the circumference of the hypotube 30′. More specifically, at least some of the flexibility enhancing features 50d′, 50i′, and 50p′ may comprise helical cuts 51′, or spiral cuts, into or through the wall 40′ of the hypotube 30′. A helical cut 51′ may extend around at least a portion of a length of the hypotube 30′ (i.e., at a non-perpendicular angle to the longitudinal axis of the hypotube 30), and may extend partially around the circumference of the hypotube 30′ (i.e., have an arc length of less than 2n, or 360°) (e.g., up 95% of the circumference of the hypotube 30′, or have an arc length of up to about 1.9n, or 342°, etc.) or completely around the circumference of the hypotube 30′ (i.e., have an arc length of 2n, or 360°, or greater), provided that helically arranged series of helical cuts 51′ does not facilitate elongation of the hypotube 30′.

A helical series of helical cuts 51′ may have the appearance of an interrupted helix, or an interrupted spiral. Solid regions between ends of helically adjacent helical cuts 51′ may define a spine 52′ of a flexibility enhancing feature 50d′, 50i′, 50p′. The spine 52′ may be oriented longitudinally, as shown in FIG. 6A, 6B, and 6C, or helically. A flexibility enhancing feature 50d′, 50i′, 50p′ may include a single spine 52′ or a plurality of spines 52′ (e.g., two spines 52′, three spines 52′, four spines 52′, etc.), depending upon the arc length each helical cut 51′ that defines the flexibility enhancing feature 50d′, 50i′, 50p′. As each spine 52′ is defined at least in part by solid regions of the hypotube 30′, each spine 52′ may resist bending. The width(s) of the spine(s) 52′, the number of spines 52′, and the arrangement of spines 52′ around the circumference of a flexibility enhancing feature 50d′, 50i′, 50p′ may at least partially dictate the column strength of the flexibility enhancing feature 50d′, 50i′, 50p′, or the ability of the flexibility enhancing feature 50d′, 50i′, 50p′ to resist compression. The width of each spine 52′ and the number of spines 52′ around the circumference of the hypotube 30′ may contribute to the flexibility/stiffness of the flexibility enhancing feature 50d′, 50i′, 50p′. The orientation of each spine 52′ on the flexibility enhancing feature 50d′, 50i′, 50p′ may determine the direction(s) in which the flexibility enhancing feature 50d′, 50i′, 50p′ may be deflected, or bend. Thus, a flexibility enhancing feature 50d′, 50i′, 50p′ may impart the catheter 10 with steerability without adding to the thickness, or outer diameter, of the catheter 10 and without decreasing the size (e.g., inner diameter, etc.) of any lumen extending through the catheter 10.

Although FIGS. 6A-6C depict the flexibility enhancing features 50d′, 50i′, and 50p′ as comprising helical cuts 51′, the hypotube 30′ may include other embodiments of flexibility enhancing features 50 (e.g., circumferential cuts, etc.) in place of or in addition to the helical 51′ cuts shown in FIGS. 6A-6C.

Turning now to FIG. 8, an embodiment of a flexibility enhancing feature 150 defined by a plurality of series 153a, 153b, 153c, etc., of cuts 151 is depicted. More specifically, each series 153a, 153b, 153c, etc., of cuts 151 of the embodiment of flexibility enhancing feature 150 depicted by FIG. 8 has a helical, or spiral configuration. As such, each series 153a, 153b, 153c, etc., may also be referred to as a “helix” or a “spiral.” The series 153a, 153b, 153c, etc., may be congruent to one another. The series 153a, 153b, 153c, etc., may share the same axis, but be translated angularly from one another around the axis. In the embodiment of flexibility enhancing feature 150 illustrated by FIG. 8, the series 153a, 153b, 153c, etc., comprise left-handed helices with a high pitch angle. Such an embodiment of flexibility enhancing feature 150 may be referred to as “multiple helix” or “multiple spiral” embodiment of flexibility enhancing feature 150.

An embodiment of a flexibility enhancing feature 150 with a plurality of helically oriented series 153a, 153b, 153c, etc., of cuts 151 may expand when the catheter 100 of which the flexibility enhancing feature 150 is a part is rotated, or torqued, in one direction (e.g., in the direction in which the helices rotate, etc.) and contract upon rotation, or torqueing, of the catheter 100 in the opposite direction (e.g., in a direction opposite from the direction in which the helices rotate, etc.). As the flexibility enhancing feature 150 expands, the cuts 151 may define thread-like ridges, which may impart the flexibility enhancing feature 150 with a screw-like shape that enables it to engage lesions, plaques, or other features that narrow the pathway through a vessel. As the flexibility enhancing feature 150 contracts, the outer diameter of the flexibility enhancing feature 150 may decrease, or tighten, which may improve the profile of the catheter 100 for crossing narrowed locations of a pathway through a vessel.

FIG. 9 illustrates an embodiment of catheter 10″ that includes a series of hypotubes 30a″, 30b″, etc., arranged end-to-end over a liner 60″ or series of liners 60″. Each hypotube 30a″, 30b″, etc., may include flexibility enhancing features 50 and, optionally, one or more operational features 55, 56.

Another embodiment of catheter 110, which includes a liner 160 on an exterior of one or more hypotubes 30, is depicted by FIGS. 10-12C. The liner 160 may comprise a polymer that lines the exterior surface 42 of the wall 40 of the hypotube 30. In a specific embodiment, the liner 160 may comprise polyethylene terephthalate (PET). More specifically, the liner 160 may comprise a contractible, or shrinkable, PET tube (e.g., a heat shrink PET tube) that has been contracted to abut the exterior surface 42 of the wall 40 of the hypotube 30 and to fit the OD defined by the exterior surface 42 of the wall 40 of the hypotube 30. Such a liner 160 may have a thickness of about 0.00025 inch (about 6.35 μm) and, thus, contribute about 0.0005 inch (about 12.7 μm) to the OD of the catheter 110.

In the embodiment depicted by FIGS. 10-12C, the liner 160 extends along a majority of a length of the catheter 110. As shown, the liner 160 may reside on the exterior surface 42 of the wall 40 of the hypotube 30 along a proximal portion 128 of the catheter 110 (FIGS. 11C and 12C) and along its intermediate portion 126 (FIGS. 11B and 12B), but the liner 160 does not reside on the exterior surface 42 of the wall 40 of the hypotube 30 along at least part of the distal portion 124 of the catheter 110 (FIGS. 11A and 12A). Thus, at the distal portion 124 of such a catheter 110, a distal portion of the hypotube 30 may remain exposed beyond a distal extent of the liner 160.

More specifically, the exterior surface 42 of the wall 40 of the hypotube 30 may be exposed at a location that includes operational features 55, 56 (FIG. 11A) (e.g., the cuts and rotating struts, respectively, of an expandable section of the hypotube 30, as disclosed by the '205 application, the '032 Design application, and the '041 Design application; etc.). The liner 160 may, in some embodiments, cover other areas of the exterior surface 42 of the wall 40 of the hypotube 30 in the distal portion 124 of the catheter 110.

In other embodiments, such an externally located liner 160 may cover an entire exterior surface 42 of a hypotube 30.

As noted previously herein, standard angiography catheters (which are typically 4 F to 6 F in size (i.e., have an OD of about 0.053 inch or 1.333 mm to about 0.079 inch or 2 mm), have pressure ratings of about 1,050 psi (about 7.25×10³ kpa) to about 1,200 psi (about 8.275×10³ kpa). Stainless steel hypotubes with the same dimensions have pressure ratings of about 8,500 psi (about 5.85×10⁴ kpa). While cuts in the hypotube may decrease the maximum pressure that can be applied to it from inside its lumen, a catheter according to this disclosure may still have a pressure rating of about 2,000 psi (about 1.4×10⁴ kpa), about 3,000 psi (about 2×10⁴ kpa), about 4,000 psi (about 2.8×10⁴ kpa), about 5,000 psi (about 3.4×10⁴ kpa), or even greater than 5,000 psi (about 3.4×10⁴ kpa). Thus, by being able to withstand increased pressure over a conventional catheter, a catheter that includes a hypotube along substantially all of its length may be safer than a conventional catheter and may provide for increased performance characteristics (e.g., higher fluid flow rates, etc.).

With returned reference to FIGS. 3A-3C, 6A-6C, and 12A-12C, in various embodiments, a method for manufacturing a catheter 10, 10′, 10″, 110 or any other embodiment of catheter according to this disclosure may include selecting and providing hypotube 30, 30′ of a desired material and dimensions (e.g., outer diameter, inner diameter, etc.). Flexibility enhancing features 50, 50′ and/or operational features 55, 56, 55′, 56′ may then be formed in or on the hypotube 30, 30′. In a specific embodiment, each flexibility enhancing feature 50, 50′ and/or operational feature 55, 55′ may be formed by defining cuts in the hypotube 30, 30′. Each cut may be performed by any of a variety of suitable processes, including, without limitation, by laser cutting techniques.

Once the flexibility enhancing features 50, 50′ and any other features have been defined in the hypotube 30, 30′, a liner 60, 60′, 160 may be applied to the hypotube 30, 30′. With reference to FIGS. 4A-4C and 7A-7C, the liner 60, 60′ may be applied to the interior surface 44, 44′ of a wall 40, 40′ of the hypotube 30, 30′, which defines a lumen 46, 46′ of the hypotube 30, 30′.

As a nonlimiting example, the liner 60, 60′ may comprise a preformed tube with outer cross-sectional dimensions, taken normal to a longitudinal axis of the liner, that enable it to be received by the inner diameter, or lumen 46, 46′, of the hypotube 30, 30′. Such a liner 60, 60′ may comprise a suitable polymer (e.g., PTFE, etc.). In some embodiments, the liner 60, 60′ may comprise a material that enables it to expand until an outer surface of the liner 60, 60′ abuts and engages the interior surface 44, 44′ of the wall 40, 40′ of the hypotube 30, 30′ (e.g., ePTFE, etc.). An outer surface of the liner 60, 60′ may be coated with a bond layer (e.g., a layer of polyether block amide (PEBA), available from Akrema S.A. of Colombes, France under the PEBAX® trademark and from Evonik Industries AG of Essen, Germany, under the VESTAMID® E trademark; etc.). The bond layer may be as thin or thinner than the liner 60, 60′ (e.g., 0.0002 inch or thinner).

Once the liner 60, 60′ is positioned within desired locations along the length of the hypotube 30, 30′, the liner 60, 60′ may be subjected to processes that will cause it to expand into, or to fit, the interior surface 44, 44′ of the wall 40, 40′ of the hypotube 30, 30′, which may secure the liner 60, 60′ to the interior surface 44, 44′ of the wall 40, 40′ of the hypotube 30, 30′. As an example, the liner 60, 60′ may be physically pressed against the interior surface 44, 44′ from within the lumen 46, 46′ (e.g., mechanically, with a long balloon, pressure vessel, etc.; under pressure; etc.). As another example, the direction of heat into a liner 60, 60′ made from an expandable material may cause the liner 60, 60′ to expand until sufficient contact is established between the outer surface of the liner 60, 60′ or a bond layer thereon and the interior surface 44, 44′ of the hypotube 30, 30′. Heat may thermally activate a bond layer on the outer surface of the liner 60, 60′, which may enable the bond layer to adhere to or bond with the interior surface 44, 44′ of the hypotube 30, 30′, securing the liner 60, 60′ to the interior surface 44, 44′.

A catheter 10, 10′ with an internal liner 60, 60′ may be manufactured from the outside-in. The inclusion of a liner 60, 60′ within the lumen 46, 46′ of the hypotube 30, 30′ may provide a surface with constant lubricity along a length of the catheter 10, 10′. By using a hypotube 30, 30′ with a liner 60, 60′ in its lumen 46, 46′ to form a catheter 10, 10′, the catheter 10, 10′ may have a small outer diameter (e.g., 6 F or less), an inner diameter that is at least as large as the inner diameter of a comparably sized microcatheter that has been manufactured from a polymer while retaining lubriciousness along its length, and a length that exceeds the length of a comparably sized microcatheter that has been manufactured from a polymer while having desired or required levels of pushability, trackability, and torqueability (e.g., 175 cm for catheters that are smaller than 6 F).

With reference to FIGS. 12A-12C, in other embodiments, a liner 160 may be applied to an exterior surface 42 of the wall 40 of a hypotube 30. Such a liner 160 may comprise a preformed tube of a contractible, or shrinkable, material (e.g., a heat shrinkable material, such as PET; etc.) with a lumen having cross-sectional dimensions, taken normal to a longitudinal axis of the liner 160, that enable it to receive the OD of the hypotube 30. The liner 160 may be cut to a desired length, and the hypotube 30 may be introduced into the lumen of the liner 160. Once the liner 160 is positioned over desired locations along the length of the hypotube 30, the liner 160 may be subjected to processes that will cause it to contract, or shrink, onto the hypotube 160, thereby securing the liner 160 to the exterior surface 42 of the wall 40 of the hypotube 30.

With the liner 60, 60′, 160 in place on the hypotube 30, 30′, the hypotube 30, 30′ may be cut to a desired length prior to forming flexibility enhancing features 50, 50′ and any other features 55, 55′, 56, 56′, prior to assembling the liner 60, 60′, 160 with the hypotube 30, 30′ or forming the liner 60, 60′, 160 on the hypotube 30, 30′, or once the liner 60, 60′, 160 has been applied to the hypotube 30, 30′.

In some embodiments, the distal end 20 of the catheter 10, 10′, 10″, 110 may be formed in a manner that preserves the inner diameter of the catheter 10, 10′, 10″, 110 or, more specifically, of its lumen 46, 46′. As an example, the edge of the hypotube 30, 30′ at the distal end 20 of the catheter 10, 10′, 10″, 110 may be smoothed, providing an opening at the distal end 20 that has an inner diameter that is the same as the inner diameter of the lumen 46, 46′.

In other embodiments, a taper may be defined at the distal end of the hypotube 30, 30′ (e.g., by altering the distal end of the hypotube, etc.) or provided at the distal end of the hypotube (e.g., by coupling a distal tip to the distal end of the hypotube, etc.). Such a taper may, by way of example only, be oriented at an angle of up to about 5° to the longitudinal axis of the hypotube. Such a taper may impart the hypotube 30, 30′ and the catheter 10, 10′, 10″, 110 of which the hypotube 30, 30′ is a part with crossability, or the ability to navigate the catheter 10, 10′, 10″, 110 through narrow restrictions in a subject's vasculature.

As an alternative to forming a taper at the distal end 20 of the catheter 10, 10′, 10″, 110, a taper may be provided at the distal end 20 by assembling a tip 70, 70′ with the distal end 20 or by forming a tip 70, 70′ on the distal end 20. For reference in describing these methods, FIGS. 13 and 14 illustrate embodiments of tips 70 and 70′, respectively, on a distal end 32 of a hypotube 30 of a catheter 10.

The tip 70, 70′ may be formed from a suitable polymer by known processes. As an example, a radiofrequency (RF) catheter tipper may be used with a tipping die and a tipping tube to form the tip 70, 70′ on the distal end 32 of the hypotube 30. The tipping tube and the distal end 32 of the hypotube 30 are introduced into the tipping die and the tipping die is heated by the RF catheter tipper. The material from which the tipping tube is formed is at least partially liquefied, and a portion of the tipping material flows into tip engagement features 34, 34′ at or near the distal end 32 of the hypotube 30. The tip engagement features 34, 34′ may mechanically anchor the tip 70, 70′ to the distal end 32 of the hypotube 30.

Tip engagement features 34 may comprise recesses, such as the series of recesses (e.g., dovetail cutouts, etc.) formed in the edge 33 of the distal end 32 of the hypotube 30, and arranged, or extending, around a circumference of the distal end 32 of the hypotube 30, as shown in FIG. 13. Tip engagement features 34′ may be formed in an exterior surface 42 of the wall 40 of the hypotube 30, adjacent to its distal end 32 (e.g., they may comprise recesses in the exterior surface 42; cuts in the wall 40 through the exterior surface 42; cutouts, or windows, through the wall 40; etc.). When the material solidifies, portions of the tip 70, 70′ that have flowed into the tip engagement features 34, 34′ in the edge 33 of the distal end 32 of the hypotube 40 forms tube engagement features 72 of the tip 70, 70′, mechanically interlocking the newly defined tip 70, 70′ and the distal end 32 of the hypotube 30.

Alternatively, the tip 70, 70′ may be formed on the distal end 32 of the hypotube 30 by a suitable molding or reflow process. A mold with a cavity that defines the tip 70, 70′ may also receive a distal portion 36 of the hypotube 30 and position the distal end 32 of the hypotube 30 adjacent to or slightly in the cavity. As a selected tip material (e.g., a polymer, etc.) may then be introduced into the cavity of the mold (e.g., as a tipper tube, in liquefied form, etc.), the liquid tip material may also extend into tip engagement features 34, 34′ in the edge 33 of the distal end 32 of the hypotube 30. As the tip material solidifies, tip material within the cavity of the mold forms the tip 70, 70′, while tip material within the tip engagement features 34, 34′ forms tube engagement features 72 of the tip 70, 70′, mechanically interlocking the tip 70, 70′ and the distal end 32 of the hypotube 30.

The tip 70, 70′ may include an opening with an inner diameter that is the same size as, substantially the same size as, or larger than the inner diameter of the lumen 46, 46′ of the hypotube 30, 30′ or a liner 60, 60′ therein (FIGS. 4A-4C and 7A-7C, respectively). An outer surface of the tip 70, 70′ may be tapered.

Suitable materials for forming the tip 70, 70′ include, without limitation, thermoplastic materials, such as PEBA. In some embodiments, such a tip 70, 70′ may include a radiopaque material, which may eliminate the need for a separate radiopaque marker. As an example, a polymer that has been infused with a radiopaque material, or throughout which particles of the radiopaque material are dispersed, may be used. Without limitation, particles of tungsten (W) may be blended into the polymer (e.g., PEBA, etc.). A specific embodiment of such a material includes 40% PEBA, by weight (w/w), and 60% tungsten, by weight (w/w).

The addition of a tip 70, 70′ to the distal end 32 of the hypotube 30 of a catheter 10 may facilitate advancement of the distal end 32 and, thus, of the catheter 10 along a desired path within a subject's body (e.g., enhance the trackability and/or crossability of the catheter 10, etc.). Interlocking between the tip 70, 70′ and the distal end 32 of the hypotube 30 may enhance torqueability of, or the ability to rotate, the hypotube 30 and the catheter 10 of which the hypotube 30 is a part.

The tip 70, 70′ may be formed or assembled with the distal end 32 of the hypotube 30 in a manner that preserves the inner diameter of the catheter 10 or more specifically, of the lumen 46 of the catheter 10. Thus, the tip 70, 70′ may not extend into the lumen 46 of the catheter 10.

Although the preceding description and the accompanying drawings are limited to a few specific embodiments, the specific embodiments that have been described and illustrated should not be construed as limiting the scope of any of the appended claims. Features from different embodiments may be employed in combination. All additions to, deletions from, and modifications of the disclosed subject matter that fall within the scopes of the claims are to be embraced by the claims.

What is claimed:

1. A catheter, comprising:
a hypotube having a length that is substantially the same as a length of the catheter, the hypotube including:
flexibility enhancing features along its length, the flexibility enhancing features having configurations and an arrangement that imparts the hypotube with desired pushability, crossability, and trackability; and
a liner substantially covering a surface of a wall of the hypotube and extending along of a majority of the length of the hypotube.

2. The catheter of claim 1, wherein the flexibility enhancing features comprise cuts in a wall of the hypotube.

3. The catheter of claim 2, wherein the cuts comprise circumferential cuts and/or helically oriented cuts.

4. The catheter of claim 1, wherein the hypotube further includes:
at least one operational feature located along at least a portion of the length of the hypotube.

5. The catheter of claim 4, wherein the at least one operational feature is located adjacent to a distal end of the hypotube.

6. The catheter of claim 4, wherein the at least one operational feature comprises cuts in a wall of the hypotube.

7. The catheter of claim 6, wherein the at least one operational feature defines a radially expandable section of the hypotube.

8. The catheter of claim 1, wherein the liner is positioned adjacent to an interior surface of a wall of the hypotube.

9. The catheter of claim 8, wherein the liner comprises an expanded polytetrafluoroethylene tube secured to the interior surface of a wall of the hypotube.

10. The catheter of claim 8, wherein the liner extends through substantially an entirety of the length of the hypotube.

11. The catheter of claim 1, wherein the liner is positioned adjacent to an exterior surface of a wall of the hypotube.

12. The catheter of claim 11, wherein the liner comprises a contracted polyethylene terephthalate tube secured to the exterior surface of a wall of the hypotube.

13. The catheter of claim 11, wherein the liner extends over substantially an entirety of the length of the hypotube.

14. The catheter of claim 11, including a distal tip defined by a distal end of the hypotube, the distal end having an opening with an inner diameter that is the same as an inner diameter of a lumen of the catheter.

15. The catheter of claim 1, further comprising:
a distal tip secured to a distal end of the hypotube in a manner that preserves an inner diameter of the catheter.

16. The catheter of claim 15, wherein the distal tip includes at least one tube engagement feature that mechanically interlocks with at least one tip engagement feature at or adjacent to a distal end of the hypotube.

17. A method for manufacturing a catheter, comprising:
defining flexibility enhancing features at a plurality of locations along a length of a hypotube; and
assembling a liner with the hypotube, the liner extending along a majority of the length of the hypotube.

18. The method of claim 17, wherein assembling the liner includes:
introducing an expandable tube into a lumen of the hypotube;
positioning the expandable tube at a desired location along the length of the hypotube; and
expanding the expandable tube until an outer surface of the expandable tube engages an interior surface of a wall of the hypotube in a manner that secures the liner within the hypotube while providing a lumen with a desired inner diameter.

19. The method of claim 17, wherein assembling the liner includes:
introducing the hypotube into a lumen of a contractible tube;
positioning the contractible tube at a desired location along the length of the hypotube; and
contracting the contractible tube until an inner surface of the contractible tube engages an exterior surface of a wall of the hypotube.

20. The method of claim 17, further comprising:
forming a tip at a distal end of the hypotube in a manner that causes the tip to mechanically engage the distal end of the hypotube while preserving an inner diameter of a lumen of the catheter, including:
causing a material from which the tip is formed to flow into at least one tip engagement feature at or adjacent to the distal end of the hypotube, the material forming at least one tube engagement feature of the tip.
